(19) United States
(12) Patent Application Publication
(10) Pub. No.: US 2004/0220498 A1
(43) Pub. Date: Nov. 4, 2004

(54) Micro medical-lab-on-a-chip in a lollipop as a drug delivery device and/or a health monitoring device

(76) Inventors: Guann-Pyng Li, Irvine, CA (US); Mark Bachman, Irvine, CA (US); Abraham Phillip Lee, Irvine, CA (US)

Correspondence Address: Daniel L. Dawes, MYERS DAWES ANDRAS & SHERMAN LLP, 11th Floor, 19900 MacArthur Boulevard, Irvine, CA, 92612, US

(21) Appl. No.: 10/763,540

(22) Filed: Jan. 23, 2004

Related U.S. Application Data
(60) Provisional application No. 60/442,220, filed on Jan. 24, 2003.

(51) Int. Cl. A61B 5/00; A61J 7/00

(52) U.S. Cl. 600/584; 600/309; 600/310; 600/349; 600/361; 600/532; 600/437; 600/476; 604/890.1; 604/77; 424/440

(57) ABSTRACT

The invention comprises an oral platform, a microchip for making physiological tests and/or delivery of drugs, and a stick connected to the platform to serve as a handle or conduit from the microchip for exterior communication. A candy shell coating on the platform incorporates medicinal agents. The platform has a plurality of fluidic ports conducive for communication of saliva to or oral delivery from the microchip. A base unit is connected to the stick and communicates to the microchip. The platform, microchip, and stick are combined together into a lollipop and further comprise a plurality of base units which are interchangeable with a plurality of lollipops. A cradle unit capable of is temporarily coupled to the base unit for recharging the base unit. The cradle unit further provides data processing, communication and/or display. The invention is also a method of making physiological tests and/or delivering drugs with the above device.

### RELATED APPLICATIONS

The present application is related to U.S. Provisional Patent Application Ser. No. 60/442,220, filed on Jan. 24, 2003, which is incorporated herein by reference and to which priority is claimed pursuant to 35 USC 119.

### BACKGROUND OF THE INVENTION

1. Field of the Invention

The invention relates to the field of biochips or micromedical-lab-on-a-chip as a drug delivery device and/or a health monitoring device.

2. Description of the Prior Art

Saliva is essential for the maintenance of oral health. It contains minerals, proteins, lipids, and immunoglobulins that are necessary for the prevention of oral diseases such as dental caries (tooth decay) and periodontal disease (destruction of the gums and the bone of the jaw that supports the teeth). This is especially apparent in cases where the saliva glands produce insufficient saliva because of disease, radiation therapy for head and neck cancer, or multiple medications with hyposalivatory side effects. Break down of the oral tissues progresses very rapidly under those circumstances. Decay (caries) can become a major and debilitating problem within months.

There has been a great deal of interest for a long time in using saliva as a mirror of systemic disease. Sampling is simpler than blood or urine, and the same components can potentially be measured. Drug compliance, monitoring of medications, pregnancy testing, hormone monitoring, virus identification, markers of HIV, bacterial infection (e.g. strep throat), cholesterol levels are all potential examples.

In the early 1990s, the fields of biochips and microfluidics were initiated in North America and Europe. Since then, there has been a revolution in molecular biology and biochemistry for drug development based on miniaturization techniques. For example, microarray gene chips have enabled the genomics field through the rapid profiling of genetic sequences to identify oncogenes and other genetic biomarkers. Another example is the development of the field of pharmacogenomics by identifying drug efficacy on single nucleotide polymorphism (SNP) through the use of capillary electrophoresis. These breakthroughs have generated a booming industry in high throughput screening based on combinatorial chemistry. By the late 1990s, point-of-care diagnostics and physiological monitoring has become the focus applications for “micro total analysis systems” technologies. Many of the microsystems have targeted body fluids such as blood and interstitial fluids, which require complex processes for preparing and purifying the samples.

In contrast, there have been relatively few reports on saliva diagnostics systems even though the sample preparation could be simpler. One of the reasons may be the tremendous challenges in attaining high signal-to-noise ratios (SNR) measurements, having influencing factors from various environmental factors (air borne pathogens, food, etc.). There is also an ongoing debate on whether whole saliva should be used versus saliva from individual glands. In addition, few technologies are available that are capable of automated sample preparation and multi-analyte detection. Thus it has been difficult to acquire sufficient information that justifies the investment in developing miniaturized systems. In this proposed research, we will pursue two types of assay systems: micro titration assays (MTA) for inorganics and microfluidic diffusion immunoassays (MDI).

Current automated titration systems are bulky devices costing nearly $10K each at present prices. These devices can perform accurate titrations, but use rather large quantities of sample and titrant and are not suited for miniaturization. The laboratory procedures for titrations and colorimetry can be readily miniaturized to chip format if fluid metering is accurate to a few percent, and the optical devices is designed to overcome Beer's Law (which dramatically reduces absorption at smaller optical path lengths). The resulting chip systems can have very broad applications beyond saliva testing, owing to the universality of the detection methods, and the broad range of needs for inorganic ion detection.

With all the advancements in lab-on-a-chip and microfluidics, there is still an ongoing need for improved biological sensors that are capable of multi-analyte detection with high sensitivity and selectivity, are compact and portable, and can operate in near real-time. Microfluidic diffusion immunoassays have been developed, but require bulky external fluidic manifolds with limitations in the number of targets that can be sensed. The sample and the antibody probe are delivered from two merging inlet microchannels resulting in a colorimetric spread at the interface of the two fluid flow lines.

Recent advancements in lab-on-a-chip research has led to a significant improvement in using a small quantity of sample and shortening the testing time to get analytical results when compared to traditional human operated procedures or robotic controlled chemistry lab approach. Current medical lab-on-a-chip devices are typically interfaced with a desk top or lap top instrument which is used to control the chips, read the sensors, display the results and store the data.

However, sample collection remains a separate step, which is typically done by using pipettes prior to injecting the samples to the micro chips. For example, the saliva tests for hormone or HIV screening are based on either the robot controlled lab or a table top instrument.

Thus to take full advantage of the micro chip for medical diagnosis applications, a seamless integration of the interface between a human subject and the micro chip instrument is crucial to the success of the lab-on-a-chip technology. Furthermore the function to delivery drugs on demand is not currently available for patient treatment.

Oral fluid tests, for example saliva tests for hormone or HIV screening, have recently been developed for chemical assays using traditional chemistry lab techniques. Current analysis techniques based on oral fluids require that a sample be collected, placed in a sealed container, mailed to a laboratory, then analyzed using traditional techniques. These techniques require the use of large laboratories and skilled technicians. The results from saliva based assays are highly dependent on the specific time that the sample was taken and the condition of the mouth at the time of the sampling. The recent advancements in medical lab-on-a-chip research (micro fluidic chips) has led to a significant improvement in using a small quantity of a testing sample and shortening the testing time to get diagnosis results over traditional chemistry lab techniques. However, sample collection remains to be a major obstacle to implement the test protocol in these micro fluidic chips.

Personalized, condition dependent drug treatment is not available in current drug delivery methods. Most methods of oral drug delivery are based on one-time test results, or on the judgment of the caregiver. Oral drug delivery is, for the most part, a one-shot approach. A single dosage is administered through the mouth, in the form of a liquid (elixir or suspension) or solid (tablet). The quantity of drug delivered is determined by the caregiver in a pre-prescribed manner.

Sustained physical monitoring of patients requires the attachment of sensors to the patient, or the diligent administering of tests by the caregiver. Neither method is popular with patients, particularly children who find it particularly uncomfortable to be subjected to tests. Sustained chemical and biochemical analysis is difficult to perform, since each test must be performed in a laboratory.

Currently, bench top or lap top instruments are the common platforms to house and control micro fluidic chips for analytical testing. These still require a skilled nurse or technician to perform the sample collection from patients and transfer the samples to the microchip instruments for testing.

### BRIEF SUMMARY OF THE INVENTION

The invention can be characterized as “a micro-laboratory that is placed within an edible coating on the end of a stick”. The best example is a laboratory in a lollipop. The invention is intended to be placed in the human mouth, but could also be placed in the mouths of animals.

A corresponding invention is “any micro-laboratory that is placed within another device whose primary function is to be placed in the mouth.” Examples of this embodiment include a laboratory in a pacifier, laboratory in a bottle nipple, laboratory in a toothbrush. This could have potential for monitoring the health of newborn babies or bottle fed animals.

The invention includes microlaboratories that can perform one test, or a multitude of tests. All manner of chemical assays are contemplated as being within the scope of the invention including those that measure the presence of a single analyte or those that perform tests on multiple analytes. For example, tests that monitor physical phenomena including temperature, viscosity, suction strength, saliva flow, mouth activity, etc. are expressly contemplated, but do not exhaust the myriad of tests which could be undertaken with the invention.

The assays that can be employed include calorimetric assays (e.g., indicators for ions or pH), absorbance, titrations, electrochemical (voltametry, amperometry, conductivity), optical scattering, immunoassays, separations including electrophoresis and chromatography.

The invention is directed to the use of a device for the operation of collecting oral fluids, including saliva. The benefit is sustained collection, higher acceptance by the subject of the collection device, and the ability to preprocess the sample during collection. For example, the fluid may pass through a filter, and be combined with preservatives during collection.

The invention is also directed to the use of a device for collecting fluids as well as the use of the device for delivering fluids. For example, a drug reservoir with an electronically controlled micropump or microejector in the case of solid or semisolid drugs, could be included in the device together with the means for collecting fluids. The device could be used for timed drug delivery pursuant to a microchip controller coupled to the micropump or microejector.

The invention is further directed to the use of a device with coatings that are designed to aid the assay, for example coatings that stimulate salivary action, coatings that stimulate a specific target response in the body, or coatings that act as calibrants to the assay.

The invention is also directed to the use of coatings to adjust the time that fluids are transferred between the mouth and the laboratory. Coatings of different thickness, density, or resistance to saliva can be used with the device.

The invention is directed to the use of multiple experiments in the device to provide redundancy over time.

The invention includes devices that are intended to induce a physical change in the subject or patient. These include heaters, electrodes, and antennas for RF microwave stimulation.

The invention includes hardware for the purpose of imaging. These include microscope in a lollipop, endoscope in a lollipop, ultrasound in a lollipop, and microwave device in a lollipop.

The invention includes an antenna in the lollipop for the purpose of wireless transmission and the use of wireless programming of the lollipop.

The invention is directed to coupling of a device with an external instrument (a “benchtop device”) designed to aid and enhance the utility of the Lollylab. For example, the external instrument could download data from the Lollylab for logging or analysis. It could also provide power and control over the laboratory. It could also draw fluid from the microlaboratory. The external instrument my use a variety of technologies to accomplish its function, including the use of its own microfluidic systems and cartridges.

The invention is used for performing diagnostics, for performing tests on populations (e.g., assessing health conditions of populations by testing for the presence of certain metals), for performing long term tests over individuals, for monitoring therapeutics, and for delivering therapeutics over time.

The invention includes but is not limited to the detection of analytes related to tooth decay or periodontal disease. Any oral assay may be considered. Oral fluid is a mirror of the blood, and can be used to monitor the condition of a subject for many purposes.

The problem solved by the invention is one of sustained data collection of oral fluids with patient acceptance (especially in children) and simplicity of application.

Therapeutic monitoring in particular is a problem that is solved by Lollylab. The Lollylab system can be used to test for the presence of a therapeutic agent (or a secondary agent that correlates to the therapy) during the course of treatment to provide information about the correct dosing and effects of therapy.

In the illustrated embodiment the invention is an apparatus for making medical diagnoses and/or delivery of drugs comprising an oral platform, a microchip mounted on or in the platform for making medical diagnoses and/or delivery of drugs, and a stick connected to the platform to serve as a handle or conduit from the microchip on the platform for exterior communication. The apparatus comprises a candy shell coating the platform with incorporated medicinal agents in the candy shell.

The platform has a plurality of fluidic ports defined therein conducive for communication of saliva to or oral delivery from the microchip.

The apparatus further comprises a base unit connected to the stick and communicated to the microchip.

The platform, microchip, and stick are combined together into a lollipop and further comprise a plurality of base units which are interchangeable with a plurality of lollipops.

The apparatus further comprises a cradle unit capable of temporarily being coupled to the base unit for recharging the base unit. The cradle unit further provides data processing, communication and/or display.

The invention is also characterized as a method for making medical diagnoses and/or delivery of drugs comprising the steps of providing an oral platform, collecting saliva or breath through the oral platform, delivering collected saliva or breath to a microchip mounted on or in the platform, and making a medical diagnosis from collected samples of saliva or breath and/or delivering drugs through the platform.

The method further comprises communicating the microchip with a base unit, including providing a plurality of platforms, microchips, and sticks as an integral units as a plurality of lollipops and interchangeably communicating a plurality of lollipops with the base unit.

The method further comprises a cradle unit capable of temporarily being coupled to the base unit for recharging the base unit. The method further comprises performing data processing, communicating data, and/or displaying data through the cradle unit from the microchip.

The step of incorporating medicinal agents in the candy shell comprises incorporating saliva producing agents in the candy shell.

The step of making a medical diagnosis from collected samples of saliva or breath comprises making the medical diagnosis entirely within the platform, microchip, and/or stick combined as an integral unit as a lollipop, or in another embodiment of making the medical diagnosis within the platform, microchip, and/or stick combined as an integral unit as a lollipop in combination with a based unit communicated to the lollipop. The method further comprises interchanging a plurality of lollipops with a base unit for making a corresponding plurality of medical diagnoses.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIGS. 1A and 1B are diagrammatic front plan views and a cross-sectional side view of the Lollylab system of the invention.

FIG. 2 is a diagrammatic front plan view of interchangeable units of the Lollylab system of the invention used in combination with a cradle.

FIG. 3 is a diagrammatic front plan view of another embodiment of the invention in which the lollipop of the invention is used in combination with a saliva diagnostics station.

FIG. 4 is a perspective cutaway view of the Lollylab of FIG. 1.

FIG. 5 is a cutaway plan view of the Lollylab of FIG. 4.

FIG. 6 is an enlarged perspective cutaway view of the Lollylab of FIGS. 4 and 5.

FIGS. 7a and 7b are diagrammatic views of a titration assay system included in a Lollylab chip of the invention.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

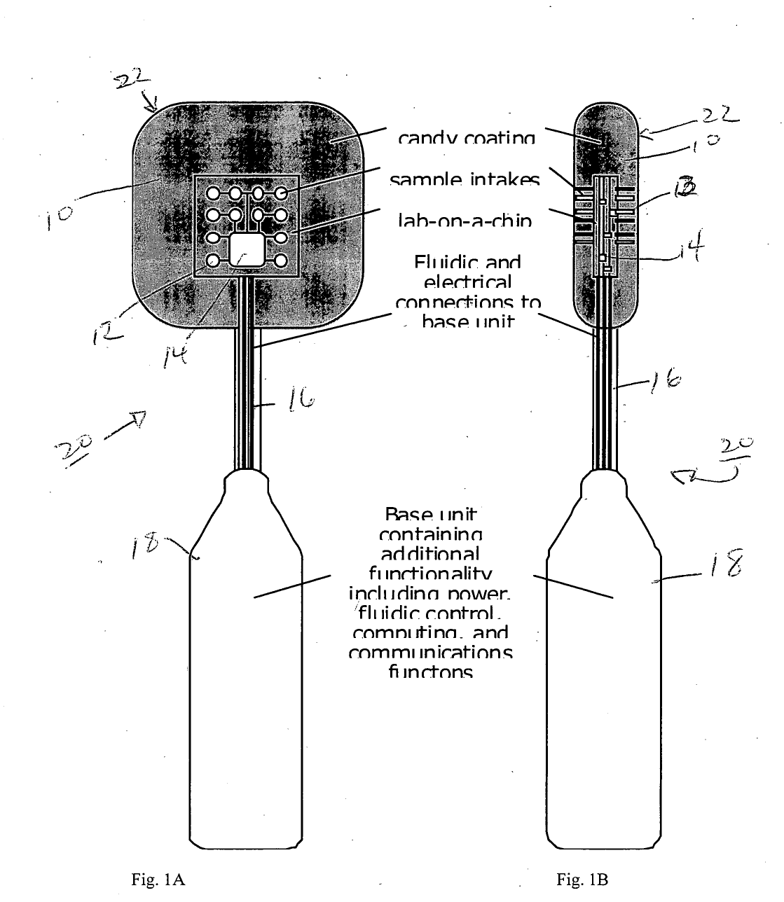

The purpose of the LollyLab™ invention provides a solution for disease diagnosis and drug delivery on demand, especially for oral and saliva-based assays and oral-based drug delivery. This Lollylab™ invention as diagrammatically depicted in FIG. 1 comprises an oral lollipop-like platform 10, such as a candy or flavor coated, lollipop-sized and smooth cornered medical instrumentation platform for housing either (1) a conventional micromedical lab-on-a-chip device 14 for monitoring health conditions, oral fluids and breath-based measurements for disease diagnoses, or (2) a conventional drug delivery microchip 14 for smart dispensing of medicines on demand.

FIG. 1 is a conceptual drawing of lab-in-a-lollipop system 20. A conventional disposable microfluidic lab chip or device 14 is embedded within a candy shell 8 on platform 10 that is formed on the end of a stick 16. The candy shell 8 may include medicinal agents to promote the operation of the chip 14, such as saliva stimulants. The chip 14 accepts saliva, or delivers fluid, from several fluidic ports or orifices 12 that become exposed as the candy shell 8 is dissolved in the patient's mouth. Depending on the chip design, the chip 14 may operate without additional support (for example, it may contain agents that change color to indicate the presence of a particular chemical, or may be a simple drug delivery mechanism). Chips 14 may interact with an optional base unit 18 if they require additional resources such as power, fluidic, light, computations or communications. Fluidic lines, electrical lines, and optical lines may be connected through the lollipop stick 16.

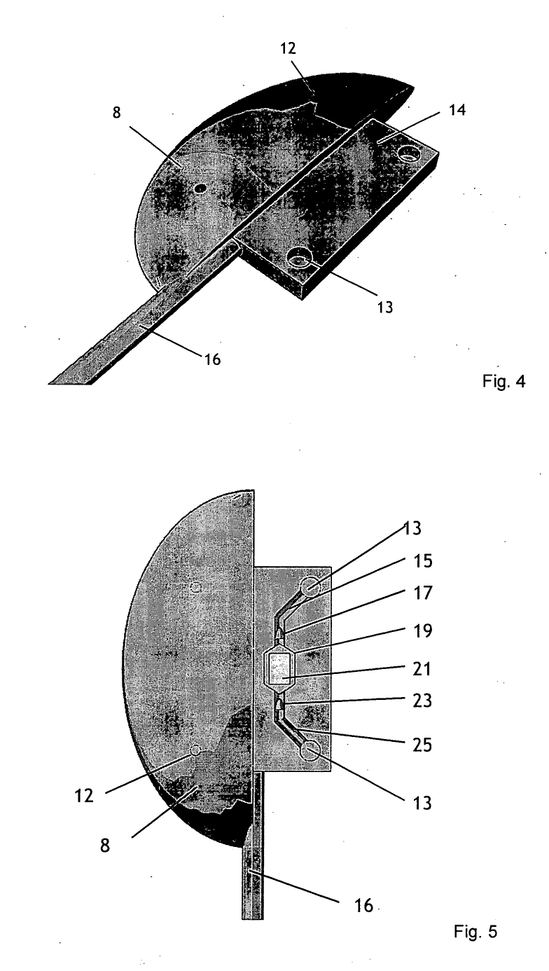

FIG. 4 is a partially cutaway perspective of Lollylab 20 showing the main elements: namely an edible or candy shell 8, orifices 12 to allow fluid to pass between chip 14 and mouth, a chip 14 containing one or more miniaturized laboratories, inlets 13 on the chip 14 with optional valves or filters to couple fluids between the microfluidic device 14 and the orifices 12 in the edible shell 8, and a rod or stick 16 designed to allow the patient to hold the Lollylab 20, and also to enable optional electrical, optical and fluidic connections between the microfluidic device 14 and external units (not shown).

FIG. 5 is a partially cutaway plan view of one embodiment of Lollylab 20 shown in FIG. 4. In this embodiment a single analytical test is embedded within Lollylab 20. It is to be expressly understood that Lollylab 20 could also include multiple test functions to test for multiple analytes. The internal microfluidic device 14 contains an inlet port 13 with a microfilter (not shown) at the opening to filter debris from the oral fluid sample. An inlet channel 15 allows fluid to pass over a miniature check valve 17 in the device 14. The fluid is directed into an internal reservoir 19. The reservoir 19 houses an absorbent material 21 that is pre-treated with a chemical indicator, such as pH indicator. Exiting the reservoir 19 is another check valve 23, which may be optional, and a fluidic channel 25 that leads back out the device 14 at a second orifice 13. The device 14 is covered with an edible shell 8 that has embedded orifices 12 to allow fluid to pass to the device 14.

Upon placing the Lollylab 20 in the mouth, the subject's saliva will dissolve the edible outer shell 8, opening up orifices 13 that lead to the microfluidic device 14. The sucking action of the subject will cause fluid to be forced through the microfluidic channel 15 by way of the one-way check valve 17. Fluid will enter the reservoir 19 and soak the pre-treated absorbent pad 21. The pad's indicator will change color in such a way to indicate the results of the test of interest. For example, the pad may change color to indicate the of pH the subject's saliva. The color may be read visually by the subject, or optionally by an embedded light sensor (not shown) in the device 14. Such an electronic sensor could be powered by conductors that are passed through the handle or stick 16. Many alternate sensors can be envisioned including those that use voltametry, amperometric or membrane specific electrodes. Other versions of this embodiment are readily envisioned, including the use of hydrophyllic channels to draw fluid without the need for check valves 17 or 25.

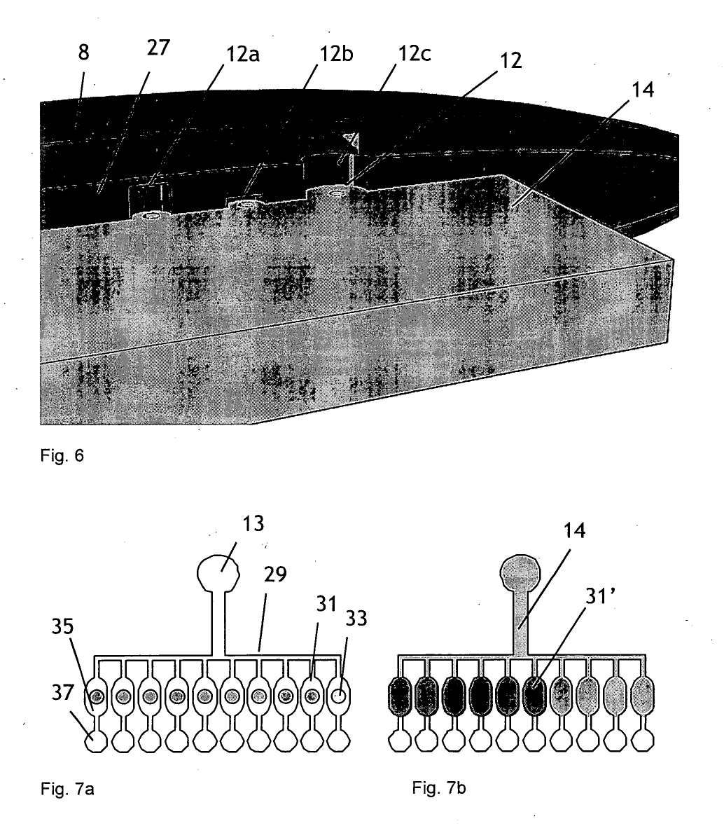

FIG. 6 is a perspective partially cutaway view of another embodiment of the Lollylab 20 of FIGS. 1-5 in which edible coating 8 may be comprised of multiple layers 27 that have different functions, for example to provide drugs or stimulus at different times. The coatings may have orifices embedded under the outer shell 8. A plurality of orifices 12a, 12b, and 12c may be placed at different depths to ensure that each opens at a different time as necessary for the intended operation of a given assay. Each orifice 12 should lead to an opening 13 in the device 14 to allow fluid to pass into or out of the microfluidic device 14.

The LollyLab™ system 20 may be designed for independent use (as a complete assay or delivery system), or as a part of a different procedure. For example, the LollyLab™ system 20 may be used to prep the mouth for oral or dental work which is intended to follow the LollyLab™ step.

The LollyLab™ surface 10 has special textures for easing the intake of fluid or vapor from the patient, and can be coated with special chemical agents to stimulate saliva glands for promoting saliva release, with agents for appropriately prepping the mouth for the assay of interest, or with chemical bonding compounds for binding with products in the exhaled air.

For the testing of bodily fluids, the lollipop 20 provides an assembly of conventional microfluidic and microelectronic chips 14 to ease the interface of the testing apparatus to the human patient, as an easy access medium for saliva sample collection from patients, and as an external fluid transporting mechanism to move oral fluids into micro-chips 14 via sucking on the lollipop 22. For pulmonary testing, the lollipop 22 may serve as a device for easing the interface of an instrument to the human patient, as an easy access medium and a concentrator for exhalation sample collection from patients, and as an air sample transporting mechanism to micro-chips 14 via inhalation and exhalation on the lollipop 22.

For disease diagnosis based on bodily fluids, the lollipop 20 will stimulate the saliva glands (or otherwise prepare the mouth), and stay in the mouth for a relatively long time, thus enabling it to make good chemical and biochemical assays (including tests for hormones, bacteria, virus, enzyme, DNA, antibody/antigen etc.) and physical measurements (including temperature, pH, salinity, viscosity, turbidity, etc.). These sets of measurements can be implemented by the conventional medical lab-on-a-chip technology currently developed at government, academic and commercial institutions. Similarly, for disease diagnosis based on pulmonary functions, the lollipop 20 will stimulate the specific chemical bonding to the exhalation contents for better detection sensitivity, and stay in the mouth for a relatively long time, thus enabling it to make good chemical/biochemical analysis (including tests for bacteria, virus, nitro-oxide, chemicals, etc.) and physical measurements (including air flow rate, breath volume, viscosity, turbidity, etc). These sets of measurements can be implemented by conventional medical lab-on-a-chip technology.

For drug delivery, the micro chips 14 may contain or deliver medicines for drug delivery on demand. Upon sucking the lollipop 20 as shown in FIG. 1, a micro-fluidic chip 14 inside the lollipop 20 can slowly release liquid medicine from reservoirs included in handle or stick 16 and coupled via lumens or tubes in neck 16 to selected ones of orifices 12 defined in surface 10, especially those drugs which are unpleasant to children (in a sugar-free version, if desired). Similarly, the lollipop 20 may provide vapor phase drugs which can be inhaled through the mouth.

The conventional microfluidic lab chip 14 may contain all the necessary reagents, drugs, electronics, power supplies, etc. to perform the operations completely within the lollipop 20 itself. It may also interface with a base unit 18 that acts as a holder for the lollipop stick 16. Electrical, fluidic and optical lines may pass from the lollipop 20, through the lollipop stick 16, to the base unit 18 for enhanced support. The base unit 18 may provide conventional fluidic control, electrical power, light sources, and dispense drugs to the lollipop chip 14. Or the base unit 18 may provide conventional chemical analysis (using other micro labchips not shown), electrical analysis and optical analysis of the oral samples. The base unit 18 may contain conventional batteries, computer chips, displays, communications systems (e.g. wireless, serial), fluidic reservoirs, photonic devices, etc.

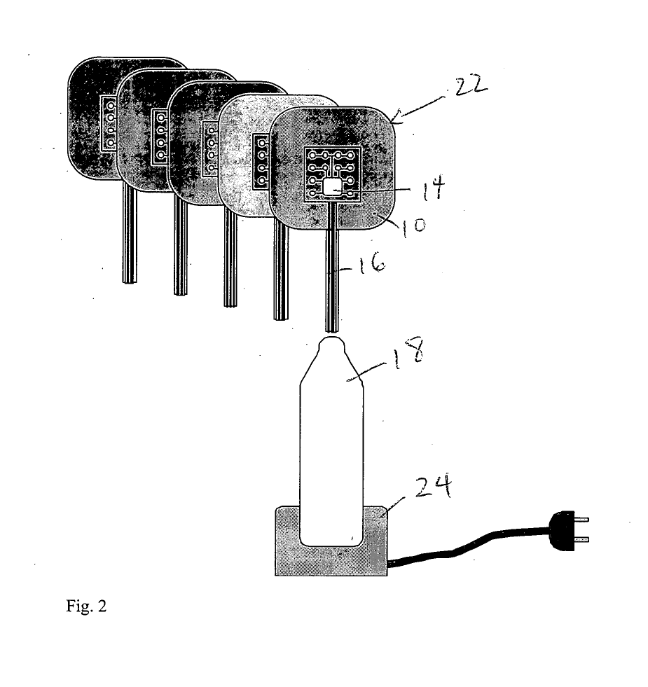

Furthermore, the base unit 18 may use a tabletop cradle 24 shown in FIG. 2 to recharge batteries, transfer data to a host computer, program the unit, or display results on a large display. In other words, as shown in FIG. 2 several different lollipops 22 and sticks 16 may be interchangeable with one or more different base units 18 depending the application which needs to be served. LollyLab microfluidic chips 14 are designed to be disposable, but may use a non-disposable base unit 18 if desired. A standard interface between the lollipop stick 16 and the base unit 18 allow multiple lab chips 14 to be interfaced to the base unit 18. Different lollipops 22 may be manufactured and distributed by different companies, and each may contain different microfluidic lab chips 14 (and different candy coatings) to perform different functions. Protocols for the lollipops 22 may be encoded within the lollipop 22 or lollipop stick 16. The base unit 18 may use a cradle 24 to recharge batteries, clean internal fluidic lines, communicate with a host computer, or display pertinent information.

The lollipop system 20 may be designed to use the base unit 18, or it may be designed to incorporate all the previously mentioned functions entirely within the lollipop 22 itself. The lollipop 20 may be a stand-alone system or it may be a part of another medical procedure (for example, it may perform mouth preparation before dental procedures). It may be used with multiple networked devices for wireless transfer the test results continuously to network hubs for monitoring time varying effects during drug treatments.

The lollipop laboratory system 20 (LollyLab™) can be used as an analytical diagnosis instrument based on oral fluid tests, exhalation tests, or physical monitoring of the mouth. The invention provides a simple solution to the collection of oral fluids, by placing the laboratory in the mouth itself. Since a lollipop 22 stimulates the saliva glands, and is likely to remain within the mouth for an extended period of time, relatively large quantities of saliva may be sampled. The extended mouth time will also result in better physical measurements (such as temperature). The LollyLabs™ 20 do not require a nurse or technician to perform the sample collection and handling, nor the testing procedure, and samples do not have to be stored in containers. In addition, the LollyLab™ system 20 can ease air sample collection from patients to examine the exhalation for contents. Even when compared to automated systems and other micro lab-on-a-chip systems, which seek to reduce the human labor and time involved in analytical testing, the current invention excels in providing efficient sampling of the fluids and vapors within the mouth, and dramatically reducing the skill required in administering the tests.

The LollyLabs™ 20 can be used as a drug delivery device for medicine, either in a liquid form to be secreted into the mouth or in a vapor form to be inhaled. This invention provides a simple, effective, and painless mechanism to slowly release drugs on demand. Since the LollyLabs™ can perform both medical diagnosis and drug delivery functions, a dosage can be adjusted based on the diagnosis of the patient's condition. This device is likely to be readily acceptable to children, and so eases the burden of delivering drugs through an extended course of treatment.

Sustained monitoring and testing is possible since the Lollylab™ system 20 is portable and may be easily used in the home environment. The high degree of acceptability by children will enable sustained measurements to be performed over the course of many minutes, hours and days. The testing may be administered by unskilled caregivers. Since the Lollylab™ system 20 has on-board electronic data collection and storage, wireless communication devices, and programmable protocols, it can enable a continuous monitoring of multiple patients over time. Thus, the Lollylab™ system 20 can facilitate large scale sampling and statistical analysis of drug treatment response for better assessment of drug performance.

The micro medical lab-on-a-chip 14 inside LollyLabs™ system 20 can perform health monitoring through the use of (1) various analytic tests of oral fluids and vapors and (2) physical measurements and sensors. It may also allow programmed drug delivery by secreting drugs into the mouth as required by the patient. The drugs may be used directly for health improvement or as one part of other mdeical tests.

The LollyLabs™ system 20 may be in hospitals, doctor's offices, long term health facilities, at home and while travelling. Significant commercial use and interest is expected in the immediate future by drug companies and the health care instrument providers. The universal nature of the Lollylab™ system 20 allows it to be used as a mini medical lab platform for a multutude of medical tests such as HIV, hormone, etc for patient care, or for drug delivery systems, each of which may be developed by a different commercial enterprise.

The invention incorporates the advancing the state-of-the-art in oral fluids-based diagnostics. As a result of the invention, oral fluids diagnostics can become a universal procedure in preventive health care and early detection of oral disorders and infectious diseases, not only in the mouth but in the rest of the human body. The preventive healthcare can be achieved by acquiring a fingerprint from saliva and identifying whether a patient is predisposed to certain health risks. A selective list of analytes is chosen to demonstrate a platform that includes several of the critical types of markers. The system is designed in a programmable fashion such that this list can be easily substituted or expanded upon based on the biological testing needs. The use of multiplexed diffusion assays open up a range of new applications not only in the area of multiplexed immunoassays, but also in the area of DNA analysis where multiplexing for high data rate analysis is also important. Furthermore, incorporation of multiplexed biomarkers such as quantum dots (QDs), enables systems capable of profiling of biomarkers for applications such as the monitoring of treatments, the development of personalized medicine, and even personalized flavors. On another front, a miniaturized, multi-analyte detection unit would be critical for biowarfare detection applications.

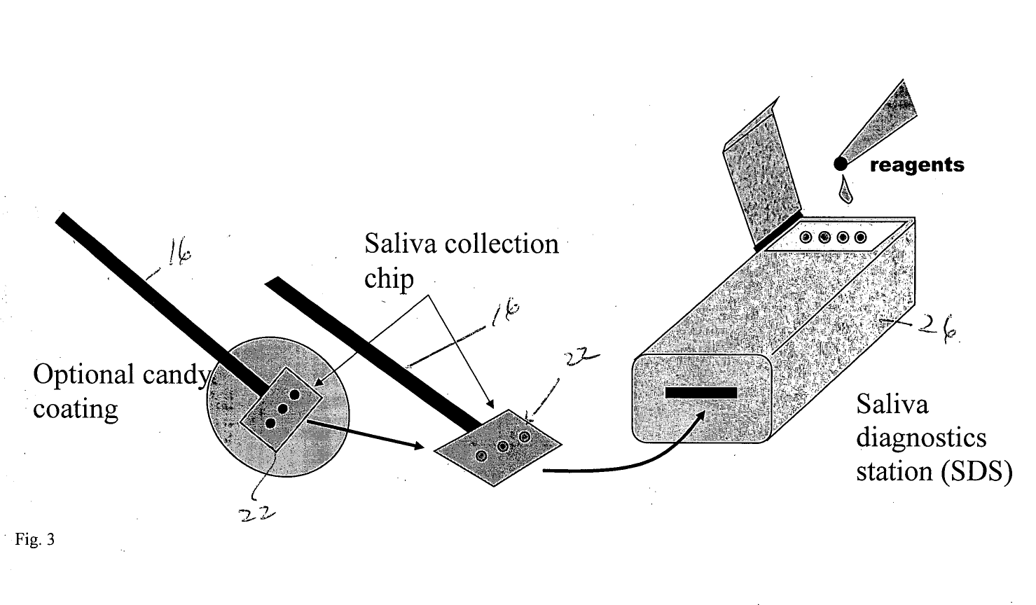

The invention achieves automated saliva sample preparation that is reconfigurable and programmable through the development of integrated microfluidic platforms 20. This is achieved by using current magnetohydrodynamic (MHD) microfluidics to provide complex fluidic routing and precision metering. Differential diffusion rates among the different saliva compounds are exploited to extract multiple targets from the solution. In the embodiment of FIG. 3 the microfluidic system is comprised of a disposable (plastic) sample collection lollipop 22 with the microchannels and electrodes, and a “saliva diagnostics station (SDS)” 26 that houses the detection components, the reagents, data acquisition, and readout.

The invention also uses multi-analyte detection schemes for saliva. The main technologies for achieving multi-analyte detection are microfluidic diffusion immunoassays (sense organic targets) and micro titration assays (sense inorganics). The micro titration assay (MTA) will be enabled by precision microfluidic metering using the MHD platform 20 to determine color changes. The microfluidic diffusion immunoassays (MDI) will be enabled by integrated microfluidic multiplexing manifolds based on magnetohydrodynamic principles.

FIGS. 7a and 7b are diagrams showing how a titration assay can be performed in a microfluidic system or device 14. As shown in FIG. 7a an inlet port 13 for collecting a sample leads to a distribution manifold 29 that directs the fluid sample into an array of small chambers 31 of fixed size. The channels leading from port 13 to chambers 31 should be hydrophilic to enable simple drawing of fluid by capillary action. Within each chamber 31 is a small absorbent pad 33 pre-soaked with a pre-determined quantity of titrant. Each pad 33 has a different quantity of titrant stored in it. Exiting each chamber 31 includes a hydrophobic vent 35 that leads to an air vent 37 to allow air to escape when the system is filled with fluid. During operation, fluid fills the device 14 and a pre-metered quantity of fluid will fill each chamber 31, equivalent to the volume of each chamber 31. The sample will not pass through the hydrophobic vent 35. The ratio of titrant to sample is fixed by the chamber volume and the initial quantity of titrant prepared in each chamber 31. As diagrammatically suggested in FIG. 7b the titration point can be determined by scanning across the array of chambers 31 to see where the appropriate chemical change has occurred, for example in chamber 31′, (e.g., phase change, precipitate, color change).

The invention uses mixed whole saliva (oral fluid) and demonstrates that components from each of the above three categories can be measured in a miniaturized laboratory for simple diagnostics during routine dental office check-ups. This specific embodiment is readily be extended to other components in the future, when particular needs are identified for clinical diagnosis, clinical trials, or clinical research.

Table 1 below lists conventional test phases in any salivary testing scheme, what the current medical practice is for each step and how this compares to the Lollylab system 20 of the invention.

TABLE 1

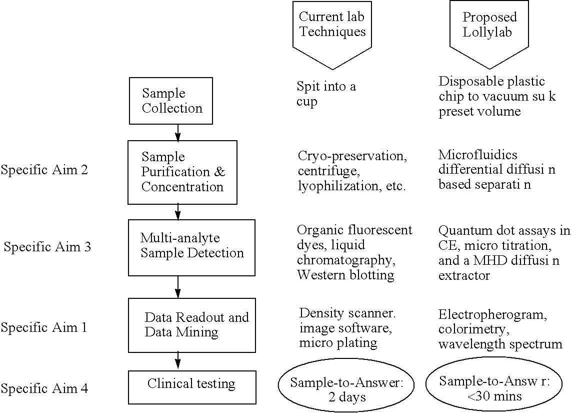

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An apparatus for making a physiological test and/or delivery of drugs comprising:
an oral platform;
a microchip mounted on or in the platform for making medical diagnoses and/or delivery of drugs; and
a stick connected to the platform to serve as a handle or conduit from the microchip on the platform for exterior communication.

2. The apparatus of claim 1 further comprising a candy shell coating the platform.

3. The apparatus of claim 2 further comprising medicinal agents in the candy shell.

4. The apparatus of claim 1 where the platform has a plurality of fluidic ports defined therein conducive for communication of saliva to or oral delivery from the microchip.

5. The apparatus of claim 1 further comprising a base unit connected to the stick and communicated to the microchip.

6. The apparatus of claim 5 where the platform, microchip, and stick are combined together into a lollipop and further comprising a plurality of base units which are interchangeable with a plurality of lollipops.

7. The apparatus of claim 6 further comprising a cradle unit capable of temporarily being coupled to the base unit for recharging the base unit.

8. The apparatus of claim 6 where the cradle unit further provides data processing, communication and/or display.

9. A method for making a physiological test and/or delivery of drugs comprising:
providing an oral platform;
collecting saliva or breath through the oral platform;
delivering collected saliva or breath to a microchip mounted on or in the platform; and
making a medical diagnosis from collected samples of saliva or breath and/or delivering drugs through the platform.

10. The method of claim 9 further comprising providing a candy shell coating the platform.

11. The method of claim 10 further comprising incorporating medicinal agents in the candy shell.

12. The method of claim 9 collecting saliva or breath through the oral platform comprises collecting saliva or breath through a plurality of fluidic ports defined therein and communicating the collected saliva or breath to the microchip or orally delivering a substance from the microchip.

13. The method of claim 9 further comprising communicating the microchip with a base unit.

14. The method of claim 13 further comprising providing a plurality of platforms, microchips, and sticks as an integral units as a plurality of lollipops and interchangeably communicating a plurality of lollipops with the base unit.

15. The method of claim 13 further comprising a cradle unit capable of temporarily being coupled to the base unit for recharging the base unit.

16. The method of claim 15 further comprising performing data processing, communicating data, and/or displaying data through the cradle unit from the microchip.

17. The method of claim 11 where incorporating medicinal agents in the candy shell comprising incorporating saliva producing agents in the candy shell.

18. The method of claim 9 where making a medical diagnosis from collected samples of saliva or breath comprise making the medical diagnosis entirely within the platform, microchip, and/or stick combined as an integral unit as a lollipop.

19. The method of claim 9 where making a medical diagnosis from collected samples of saliva or breath comprise making the medical diagnosis within the platform, microchip, and/or stick combined as an integral unit as a lollipop in combination with a based unit communicated to the lollipop.

20. The method of claim 19 further comprising interchanging a plurality of lollipops with a base unit for making a corresponding plurality of medical diagnoses.

21. A micro-laboratory for oral insertion to collect oral fluids comprising:
a microfluidic device for analyzing the oral fluids;
an edible coating disposed on the microfluidic device; and
a handle coupled to the microfluidic device.

22. The micro-laboratory of claim 21 further comprising an oral device to be placed in the mouth combined with the microfluidic device to facilitate oral use.

23. The micro-laboratory of claim 22 where the oral device comprises a pacifier, a bottle nipple, or a toothbrush.

24. The micro-laboratory of claim 21 where the microfluidic device performs a plurality of tests, including chemical assays that measure the presence of a single analyte or multiple analytes.

25. The micro-laboratory of claim 21 where the microfluidic device performs tests that monitor physical phenomena including temperature, viscosity, suction strength, saliva flow, or mouth activity.

26. The micro-laboratory of claim 21 where the microfluidic device performs assays that include colorimetric assays (e.g., indicators for ions or pH), absorbance, titrations, electrochemical (voltametry, amperometry, conductivity), optical scattering, immunoassays, or separations including electrophoresis and chromatography.

27. The micro-laboratory of claim 21 where the microfluidics device collects saliva, whereby sustained collection, higher acceptance by a patient of collection, and the ability to preprocess the sample during collection is provided.

28. The micro-laboratory of claim 27 further comprising a filter and preservation means for preserving the saliva, where the saliva passes through the filter and is combined with preservatives by the preservation means during collection.

29. The micro-laboratory of claim 21 further comprising means for delivering drugs.

30. The micro-laboratory of claim 29 where the means for delivering drugs is controlled to provide timed drug delivery.

31. The micro-laboratory of claim 21 where the coating is adapted to aid an assay performed by the microfluidics device.

32. The micro-laboratory of claim 31 where the coating stimulates salivary action, stimulates a specific target response in the body, or acts as a calibrant to the assay.

33. The micro-laboratory of claim 31 where the coating adjusts the time that fluids are transferred between the mouth and the microfluidics device by means of different thicknesses, densities, or resistance to saliva of the coating.

34. The micro-laboratory of claim 21 further comprising a kit of multiple micro-laboratories for use in a corresponding multiple of tests to provide redundancy over time.

35. The micro-laboratory of claim 21 further comprising means for inducing a physical change in a patient.

36. The micro-laboratory of claim 35 where the means for inducing a physical change in a patient comprises a heater, one or more electrodes, or an antenna for RF microwave stimulation.

37. The micro-laboratory of claim 21 further comprising means for imaging.

38. The micro-laboratory of claim 37 where the means for imaging comprise a microscope, an endoscope, an ultrasound imaging device, or a microwave imaging device.

39. The micro-laboratory of claim 21 further comprising an antenna for wireless transmission and wireless programming of the microfluidics device.

40. The micro-laboratory of claim 21 further comprising an external instrument designed to aid and enhance the utility of the micro-laboratory such as downloading data from the microfluidics device for logging or analysis, to provide power and control over the microfluidics device, or to draw fluid from the microfluidics device.

41. The micro-laboratory of claim 21 where the microfluidics device performs diagnostics, performs population tests, performs long term tests, monitors therapeutics, or delivers therapeutics over time.

42. The micro-laboratory of claim 21 where the microfluidics device detects analytes related to tooth decay or periodontal disease.

43. The micro-laboratory of claim 21 where the microfluidics device is used for sustained data collection of oral fluids with patient acceptance and simplicity of application.

44. The micro-laboratory of claim 21 where the microfluidics device is used to test for the presence of a therapeutic agent or a secondary agent that correlates to a therapy during the course of treatment to provide information about the correct dosing and effects of therapy.
